(19) United States
(12) Patent Application Publication
(10) Pub. No.: US 2024/0215838 A1
(43) Pub. Date: Jul. 4, 2024

(54) APPARATUS FOR TAKING BLOOD PRESSURE MEASUREMENTS AND DISPLAYING THE SAME

(71) Applicant: GE Precision Healthcare LLC, Wauwatosa, WI (US)

(72) Inventors: Juha Virtanen, Helsinki (FI); Matti Laitinen, Helsinki (FI); Lawrence R. Boyd, Milwaukee, WI (US); Faye L. Aebly, Germantown, WI (US); René Coffeng, Helsinki (FI); Emma Hellman, Espoo (FI)

(21) Appl. No.: 18/149,892

(22) Filed: Jan. 4, 2023

(51) Int. Cl. A61B 5/0215; A61B 5/00

(52) U.S. Cl. CPC A61B 5/0215; A61B 5/0022

(57) ABSTRACT

An apparatus comprising a housing, a detection system, and a transmission system. The detection system is disposed within the housing. The detection system is configured to measure a blood pressure of a patient. The transmission system is disposed within the housing. The transmission system is operatively coupled to the detection system and is configured to transmit a signal indicative of a blood pressure of the patient.

### TECHNICAL FIELD

This disclosure relates generally to blood pressure measurement devices, and more specifically, to invasive blood pressure measurement devices and display systems.

### BACKGROUND

Blood pressure measuring devices are frequently used to measure the blood pressure of an individual, such as a patient in a hospital or doctor's office. For example, invasive blood pressure measuring devices may be used in a hospital or surgical setting. Such blood pressure measuring devices may be operatively coupled to a display device via a wire or a cable. In some cases, in such hospital or surgical settings there may be many different wires, cables, tubes, and other connection members laying on the floor of the hospital or surgical room. In some cases, the wire or cable that operatively couples the blood pressure measuring device to the display device may contribute to the many wires, cables, connection members on the floor of the hospital or surgical room.

### SUMMARY

In a general aspect, an apparatus comprises a housing, a detection system, and a transmission system. The detection system is disposed within the housing. The detection system is configured to be operatively coupled to an artery or a vein of a patient and to measure a blood pressure of the patient. The transmission system is disposed within the housing. The transmission system is operatively coupled to the detection system and is configured to transmit a wireless signal indicative of a blood pressure of the patient.

In another aspect, an apparatus comprises a housing, a detection system, a transmission system, a battery, and an activation switch. The housing defines a cavity. The detection system is disposed within the cavity of the housing. The detection system includes a pressure transducer and is configured to measure a blood pressure of a patient. The transmission system is disposed within the cavity of the housing. The transmission system is operatively coupled to the detection system and is configured to transmit a wireless signal indicative of a blood pressure of the patient. The battery is disposed within the cavity of the housing and is operatively coupled to the detection system and to the transmission system. The activation switch is operatively coupled to the battery. At least a portion of the activation switch is disposed outside of the cavity of the housing.

In another aspect, a system comprises a sensor or measuring device and a display device. The measuring device has a housing, a detection system, and a transmission system. The detection system is disposed within the housing. The detection system is configured to be operatively coupled to an artery of a patient and to measure a blood pressure of the patient. The transmission system is disposed within the housing. The transmission system is operatively coupled to the detection system and is configured to transmit a wireless signal indicative of a blood pressure of the patient. The display device has a transmission system and a display. The transmission system of the display device is configured to receive the wireless signal indicative of a blood pressure of the patient from the transmission system of the measuring device. The display is configured to display a blood pressure of the patient.

### BRIEF DESCRIPTION OF THE DRAWINGS

FIG. 1 is a block diagram of an apparatus according to an aspect.

FIG. 2 illustrates a system according to an aspect.

FIG. 3 is a block diagram of the system of FIG. 2.

FIG. 4 is a block diagram of a portion of a system according to an aspect.

FIG. 5 is a perspective view of the measuring device or sensor of the system of FIG. 2.

FIG. 6 illustrates a portion of the system.

FIG. 7 is a block diagram of a system according to an aspect.

FIG. 8 is a perspective view of a coupling member according to an aspect.

FIG. 9 is a block diagram of a system according to an aspect.

### DETAILED DESCRIPTION

In general, the implementations are directed to medical devices and methods. The term patient or user may hereinafter be used for a person who benefits from the medical device or the methods disclosed in the present disclosure. For example, the patient can be a person whose body contacted by, engaged with, or otherwise interacts with the medical device.

In some implementations, an apparatus or device is configured to measure a blood pressure of a patient and wirelessly transmit a signal indicative of the blood pressure of a patient to a display unit, such as a visual display unit. Accordingly, in such implementations, the apparatus or device is devoid of a wire extending from the device that measures the blood pressure of the patient to the device that displays the blood pressure of the patient. Accordingly, in some aspects, the wireless nature of the system may facilitate the movement of a patient from one location to another location.

In some implementations, the apparatus or device is configured to invasively measure the blood pressure of the patient. For example, in some implementations, the apparatus or device is operatively coupled to an artery or a vein of the patient to facilitate the blood pressure measurement of the patient. In some implementations, the apparatus or device includes a housing, such as a single housing, that is configured to house a power system, a blood pressure detection system, and a transmission system.

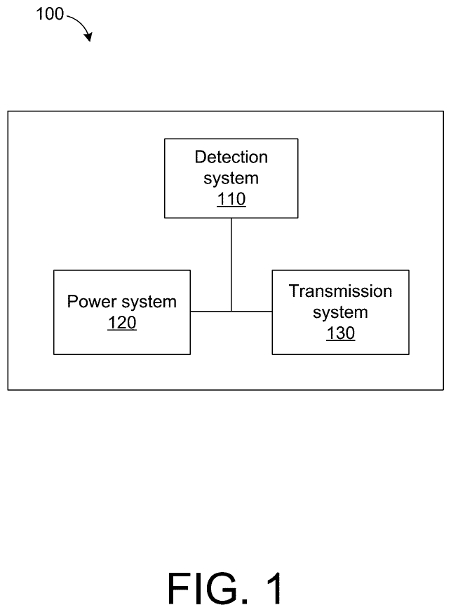

FIG. 1 is a block diagram of an apparatus 100 according to an aspect. The apparatus 100 includes a detection system 110, a power system 120, and a transmission system 130. The apparatus 100 may be operatively coupled to a body of a patient and may detect or measure the blood pressure of the patient. The apparatus 100 may transmit the blood pressure of the patient to a display or display unit for display of the measured blood pressure. For example, the apparatus 100 may send a wireless signal indicative or representative of the blood pressure of the patient to a display or display unit.

In some aspects, the apparatus 100 includes a housing and the detection system 110, the power system 120, and the transmission system 130 are disposed with the housing. For example, a housing may define a cavity and the detection system 110, the power system 120, and the transmission system 130 may be disposed within the cavity of the housing.

The detection system 110 may be configured to detect or measure a blood pressure of a patient. In some aspects, the detection system 110 may be an invasive blood pressure measuring system and may be operatively coupled to an artery of a patient. In some aspects, the detection system 110 includes a pressure transducer that is configured to be operatively coupled to a patient, such as to an artery or to a vein of the patient, to detect and measure the blood pressure of the patient.

The transmission system 130 may be operatively coupled to the detection system 110. The transmission system 130 is configured to transmit a wireless signal indicative of the blood pressure of the patient as detected or measured by the detection system 110. For example, in some aspects, the transmission system 130 is configured to send a radiofrequency (RF) signal to a display unit. Accordingly, in some aspects, the transmission system 130 includes a radiofrequency transmitter.

The power system 120 is configured to provide power to the apparatus 100. In the illustrated aspect, the power system 120 is operatively coupled to the detection system 110 and to the transmission system 130. In some aspects, the power system 120 includes a battery, such as a zinc-air battery or other type of battery or power source.

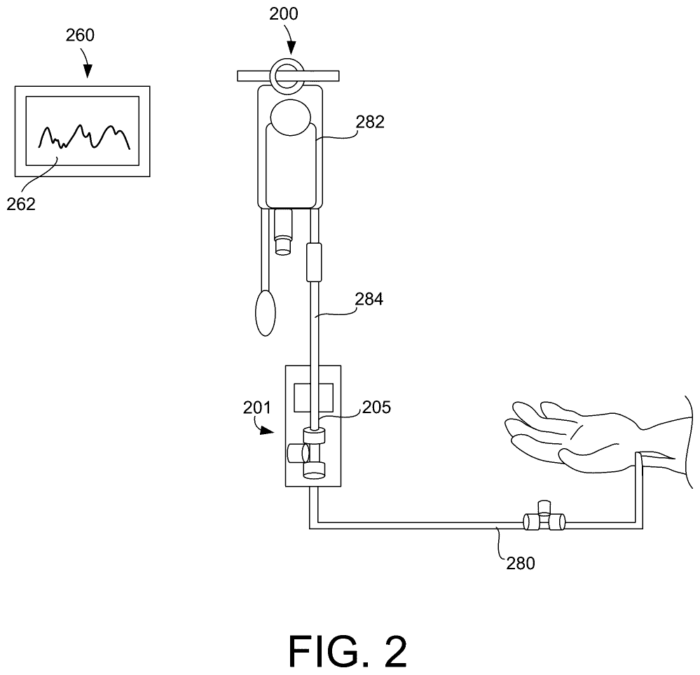

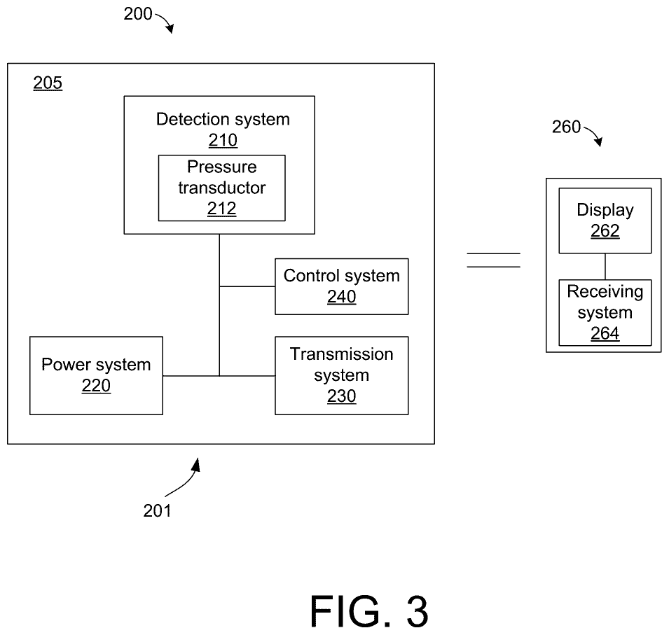

FIG. 2 illustrates a system or apparatus 200 according to an aspect. FIG. 3 is a block diagram of the system or apparatus 200. The apparatus 200 may be configured to measure and display a blood pressure of a patient. In some implementations, the apparatus, 200 may be configured to invasively measure the blood pressure of the patient. As best illustrated in FIG. 2, in some implementations, the apparatus 200 may be operatively coupled to an artery or vein of a patient. For example, the apparatus 200 may be operatively coupled to an artery in an arm or wrist of a patient. In other implementations, the apparatus 200 may be operatively coupled to an artery at a different location within the body of the patient. In yet other implementations, the apparatus 200 may be operatively coupled to a vein of the patient.

As best illustrated in FIG. 2, the system or apparatus 200 includes a measuring device or sensor 201, a display system 260, a tubular member 280 extending between the housing 205 and the patient, a pressure bag 282, and a tubular member 284 extending between the pressure bag 282 to the housing 205. As best illustrated in FIG. 3, the system or apparatus also includes a detection system 210, a power system 220, a transmission system 230, and a control system 240 disposed within the housing 205. Additionally, a display system 260 includes a visual display 262 and a receiving system 264.

In some aspects, the system or apparatus 200 is configured to display, such as visually display, the blood pressure measurement of the patient on the display 262. In some aspects, the blood pressure detection system 210 is configured to wirelessly communicate with the display system 260 to communicate the blood pressure measurement of the patient.

The sensor 201 includes a detection system 210, the power system 220, the transmission system 230, and the control system 230 are disposed with the housing 205. For example, the housing 205 may define a cavity and the detection system 210, the power system 220, the transmission system 230, and the control system 240 are disposed within the cavity of the housing 205.

The detection system 210 is configured to detect or measure a blood pressure of a patient. In the illustrated aspect, the detection system 210 is an invasive blood pressure measuring system. Accordingly, the detection system 210 is operatively coupled to an artery or a vein of a patient. As illustrated in FIG. 3, the detection system 210 includes a pressure transducer 212 that is configured to be operatively coupled to a patient, such as to an artery of the patient, and to detect and measure the blood pressure of the patient.

The transmission system 230 is operatively coupled to the detection system 210. The transmission system 230 is configured to transmit a wireless signal indicative of the blood pressure of the patient as detected or measured by the detection system 210. For example, in some aspects, the transmission system 230 is configured to send a radiofrequency (RF) signal to a display unit or system 260. Accordingly, in some aspects, the transmission system 130 includes a radiofrequency transmitter. In other aspects, the transmission system 230 is configured to send a different type of wireless signal, such as a Bluetooth signal, to the display unit or system 260. In some aspects, the transmission system 230 includes a transmission antenna or other device that is configured to transmit wireless signals.

The power system 220 is configured to provide power to the apparatus 200. In the illustrated aspect, the power system 220 is operatively coupled to the detection system 210 and to the transmission system 230. In some aspects, the power system 220 includes a power source such as a battery. In some aspects, the battery may be a zinc-air battery or other type of battery.

In the illustrated implementation, the housing 205 also houses a control system 240. The control system 240 is operatively coupled to the detection system 210, the power system 220 and the transmission system 230. The control system 240 is configured to control the device and the components of the device. For example, in some implementations, the control system 240 includes a memory and a processing system. In some implementations, an executable program is stored in the memory. In some implementations the control system 240 includes an analog front-end component.

The display unit 260 includes a receiving system 264 and a display 262. The receiving system 264 is configured to receive signals from the transmission system 230 indicative of the blood pressure of the patient. In some aspects, the receiving system 264 includes an antenna or other device that is configured to receive wireless signals. The receiving system 264 is operatively coupled to the display 262. The display 262 is configured to visually display the measured blood pressure of the patient. For example, the display may be a light emitting diode display, a liquid crystal display, or another type of display.

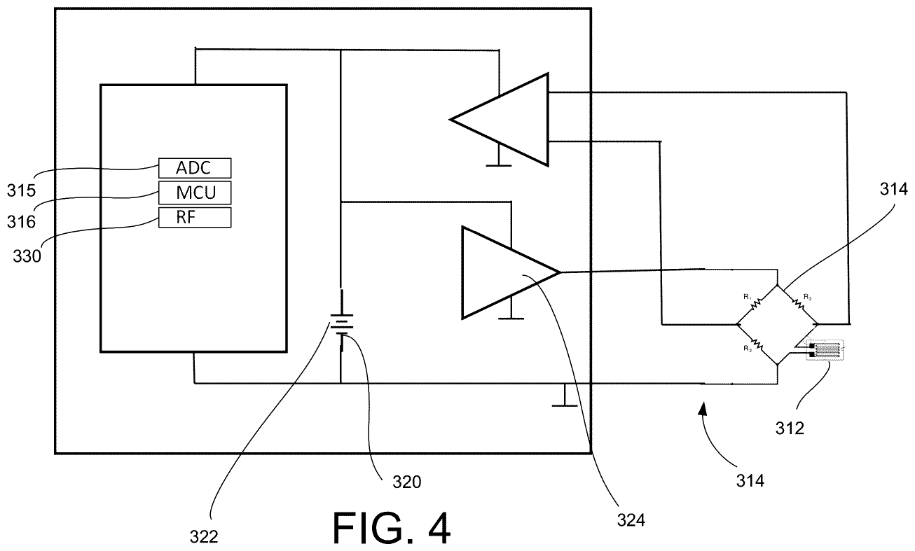

FIG. 4 schematically illustrates a portion of a sensor according to an aspect. The sensor includes a power system 320, a detection system 310, a transmission system 330, an analog to digital unit (ADC) 315, and a microcontroller unit (MCU) 316 according to an aspect. In the illustrated aspect, the power system 320 includes a battery 322 that is operatively coupled to the detection system 310 and to the transmission system 330. The sensor includes a driver 324 that supplies power or voltage from the battery 322 to a strain gauge 312 and a bridge 314 of the detection system. The detection system 310 is configured to detect and measure the blood pressure of the patient. The sensor also includes an amplifier 324 that amplifies the signal from the bridge 314. The analog to digital unit (ADC) 315 digitizes the signal. The microcontroller unit (MCU) 316 processes the signal and sends the signal to the transmission system 330. In the illustrated embodiment, the transmission system 330 includes a radio frequency (RF) transmitter.

In some systems, a typical sample rate for the digitized signal is 250 samples per second. In some such systems, the impedance of the strain gauge bridge is about 400 ohms. Additionally in some such systems, the bridge is powered using continuous AC or DC voltage, the amplitude of which is about 4 Vrms. With these values, the bridge consumes 40 mW of power continuously.

In some aspects of the invention, the bridge powering scheme may be varied to lengthen the life of the battery 322. For example, the bridge power can be turned on only when the bridge voltage is sampling. A sample rate of 250 samples per second corresponds to 4 ms time period between samples. If the sampling time is for example 100 us and the bridge power is kept on only during data sampling, the power consumption of the bridge is reduced by factor of 100/4000, i.e. from 40 mW to 1 mW when compared to other systems. In some examples, the battery is configured to last about 3 days. In other implementations, the battery is configured to last longer than 3 days.

In some aspects, the bridge impedance may be increased to lengthen the life of the battery 322. For example, the bridge impedance can be designed to be as high as 10,000 ohms. When compared to other systems, increasing the bridge impedance from 400 ohms to 10,000 ohms decreases power consumption by factor of 400/10000, i.e. from 4 mW to 1.6 mW.

In some aspects, the impedance of the bridge may be larger because the battery powered device is less sensitive to the surrounding environment because there are less wires in close proximity to the device.

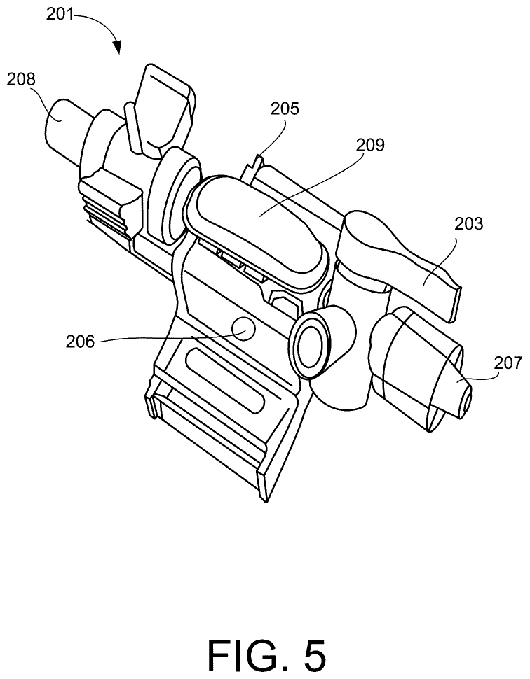

FIG. 5 is a perspective view of the measuring device or sensor 201. The sensor 201 includes the housing 205. The housing has an outer surface 209 and defines an inner cavity. All of the components of the measuring device or sensor 201 (the detection system, the power system, the transmission system, and the control system) are disposed within the cavity defined by the housing 205. The housing defines a first port 207 and a second port 208.

The first port 207 is configured to engage with and be coupled to an end portion of the tubular member 280 that extends between and operatively couples the sensor 201 to the patient. For example, the tubular member 280 may extend between and operatively couple the sensor 201 to an artery of the patient. In other aspects, the tubular member 280 may extend between and operatively couple the sensor 201 to a different portion of the patient, such as a vein of a patient. The second port 208 is configured to engage with and be coupled to an end portion of the tubular member 284 that extends between and operatively couples the sensor 201 to the pressure bag or fluid reservoir 282.

The sensor 201 includes an activation or on/off switch 206. In the illustrated implementation, the activation or on/off switch 206 is disposed on the outer surface 209 of the housing 205. For example, in some aspects, at least a portion of the activation or on/off switch is disposed outside of the housing or on an outer surface of the housing. The activation or on/off switch is operatively coupled to the components of the sensor 201. For example, in some aspects, the activation or on/off switch 206 may be used to activate or turn on (or turn off) the detection system of the device. In such aspects, the battery power of the power system 220 may be conserved when the sensor 201 is not in use.

In the illustrated implementation, the sensor 201 includes a valve 203. The valve 203 may be used to slow or stop the exchange of fluid between the sensor 201 and the patient. Accordingly, the valve 203 may be used to slow or stop the flushing of the system. In some implementations, the valve 203 is coupled to the housing 205. For example, in some implementations, the valve 203 may be pivoted or rotated with respect to the housing 205 but is otherwise coupled to the housing 205.

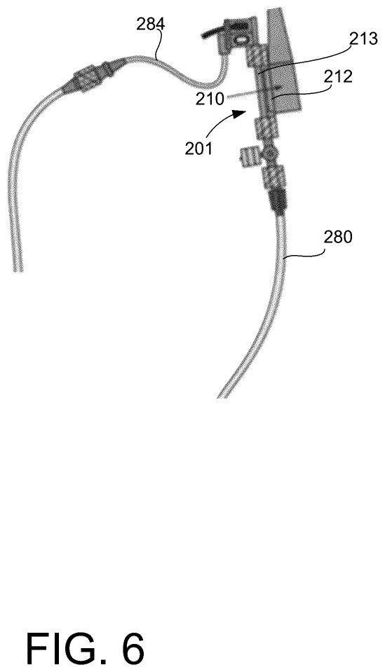

FIG. 6 illustrates the sensor 201 in use. In the illustrated implementation, the tubular member 280 extends between and operatively couples the sensor 201 to an artery or vein of the patient. The tubular member 284 extends between and operatively couples the sensor 201 to the pressure bag or fluid reservoir. As illustrated, the pressure bag or fluid reservoir is fluidically coupled to the artery or vein of the patient via a line 213 within the sensor 201. This arrangement allows the pressure bag or fluid reservoir to slowly provide fluid to the patient. In some implementations, this arrangement helps prevent coagulation of the blood of the patient and inaccurate blood pressure measurements. In the illustrated implementation, the detection system 210 including the pressure transducer 212 is operatively coupled to the body (the artery or the vein) of the patient. As illustrated, the pressure transducer 212 is disposed proximate or at the side of the line 213 within the sensor 201 that includes the blood of the patient and is fluidically coupled to the artery or vein of the patient. According, the pressure transducer 212 is disposed such that the pressure transducer 212 interfaces or interacts with the line or conduit 213 that includes the blood of the patient.

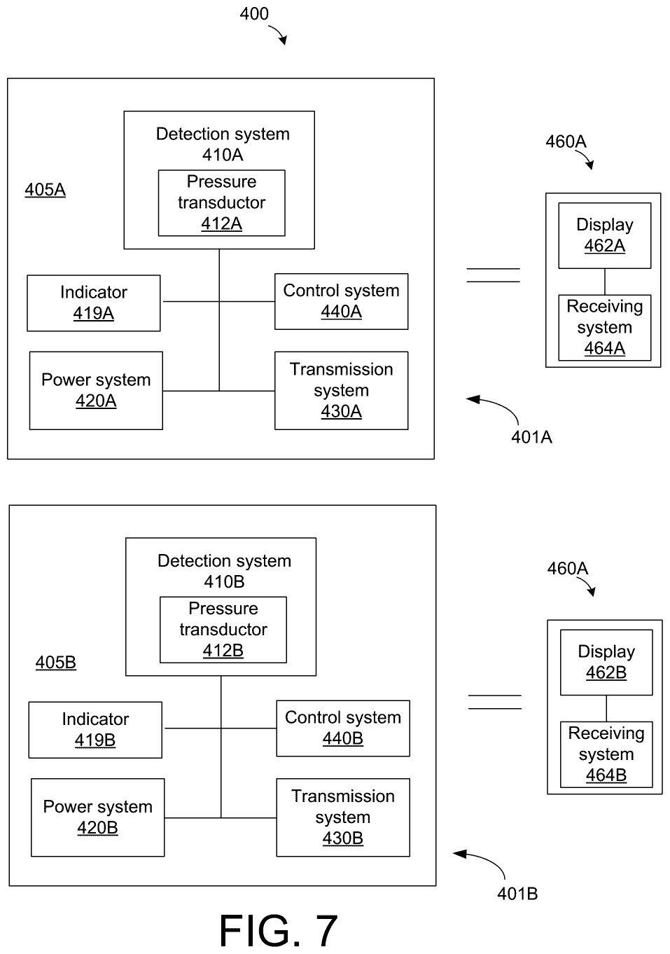

FIG. 7 schematically illustrates an apparatus 400 according to an implementation. The apparatus includes a first sensor 401A, a second sensor 401B, a first display 460A, and a second display 460B.

In some aspects, the apparatus 400 is configured to display or transmit for display, such as visually display, the different types of blood pressure measurements of the patient. In some implementations, the apparatus 400 is configured to display, such as visually display, more than one blood type of blood pressure measurement of the patient at the same time. For example, in some implementations, the apparatus 400 is configured to measure and display an arterial blood pressure of the patient and a venous blood pressure of the patient.

The first sensor 401A has a detection system 410A, a power system 420A, a transmission system 430A, and a control system 440A disposed within a housing 405A. In some aspects, the first sensor 401A or components of the first sensor 401A are configured to wirelessly communicate with the first display system 460A to display the blood pressure measurements of the patient.

For example, the housing 405A may define a cavity and the detection system 410A, the power system 420A, the transmission system 430A, and the control system 440A are disposed within the cavity of the housing 405A. In some implementations, the detection system 410A is an invasive blood pressure measuring system. Accordingly, in such implementations, the detection system 410A is physically and operatively coupled to a portion of the body of the patient. For example, the detection system 410A may be operatively coupled to an artery of the patient. In such implementations, the detection system 410A is configured to detect and measure the arterial blood pressure of the patient. As illustrated in FIG. 6, in the illustrated implementation, the detection system 410A includes a pressure transducer 412A that is configured to be operatively coupled to the patient, such as to an artery of the patient, and to detect and measure the arterial blood pressure of the patient.

The transmission system 430A is operatively coupled to the detection system 410A. The transmission system 430A is configured to transmit a wireless signal indicative of the blood pressure of the patient as detected or measured by the detection system 410A. For example, in some aspects, the transmission system 430A is configured to be paired with the display unit 460A. In some implementations, the transmission system 430A may be paired with the display unit 460A based on proximity between the transmission system 430A and display unit 460A. The devices may use electric or magnetic fields, such as near field communication (NFC), radio frequency identification (RFID), Bluetooth or similar means for pairing. In other aspects, the transmission system 430A is configured to send a different type of wireless signal to the display unit or system 460A.

The power system 420A is configured to provide power to the detection system 410A. In the illustrated aspect, the power system 420A is operatively coupled to the detection system 410A and to the transmission system 430A. In some aspects, the power system 420A includes a battery, such as a zinc-air battery or other type of battery.

In the illustrated implementation, the housing 405A also houses a control system 440A. The control system 440A is operatively coupled to the detection system 410A, the power system 420A, and the transmission system 430A. The control system 440A is configured to control the device and the components of the device. For example, in some implementations, the control system 440A includes a memory and a processing system. In some implementations, an executable program is stored in the memory. In some implementations the control system 440A includes an analog front-end component.

The display unit 460A includes a receiving system 464A and a display 462A. The receiving system 464A is configured to receive signals from the transmission system 430A indicative of the blood pressure of the patient. For example, when the detection system 410A is operatively coupled to an artery of the patient, the receiving system 464A receives signals from the transmission system 430A indicative of the arterial blood pressure of the patient. In some aspects, the receiving system 464A includes an antenna or other device that is configured to receive wireless signals. The receiving system 464A is operatively coupled to the display 462A. The display 462A is configured to visually display the measured blood pressure of the patient (such as the arterial blood pressure of the patient). For example, the display may be a light emitting diode display, a liquid crystal display, or another type of display. In some implementations the display unit 460A is configured to provide a visual indication as to what type of blood pressure is being displayed or which sensor the display unit 460A is receiving information from. For example, in some implementations the display 462A may provide such visual indication.

In some implementations, the sensor 401A includes an indicator 419A. The indicator 419A is configured to provide a visual indication as to which display system the sensor 401A is paired with. For example, the indicator 419A may include a light emitting diode or a series of light emitting diodes that provide a visual indication as to which display system the sensor 401A is paired with. In some examples, wireless communication and the indicator 419A allow a user or a physician to know what display the sensor 401A is communicating with without following a wire.

The second sensor 401B has a detection system 410B, a power system 420B, a transmission system 430B, and a control system 440B disposed within a housing 405B. In some aspects, the second sensor 401B or components of the second sensor 401B are configured to wirelessly communicate with the second display system 460B to display the blood pressure measurements of the patient.

The housing 405B may define a cavity and the detection system 410B, the power system 420B, the transmission system 430B, and the control system 440B are disposed within the cavity of the housing 405B. In some implementations, the detection system 410B is an invasive blood pressure measuring system. Accordingly, in such implementations, the detection system 410B is physically and operatively coupled to a portion of the body of the patient. For example, the detection system 410B may be operatively coupled to a vein of the patient. In such implementations, the detection system 410B is configured to detect and measure the venous blood pressure of the patient. As illustrated in FIG. 7, in the illustrated implementation, the detection system 410B includes a pressure transducer 412B that is configured to be operatively coupled to the patient, such as to a vein of the patient, and to detect and measure the venous blood pressure of the patient.

The transmission system 430B is operatively coupled to the detection system 410B. The transmission system 430B is configured to transmit a wireless signal indicative of the blood pressure of the patient as detected or measured by the detection system 410B. For example, in some aspects, the transmission system 430B is configured to be paired with the display unit 460B. In some implementations, the transmission system 430B may be paired with the display unit 460B based on proximity between the transmission system 430B and display unit 460B. The devices may use electric or magnetic fields, such as near field communication (NFC), radio frequency identification (RFID), Bluetooth or similar means for pairing. In other aspects, the transmission system 430B is configured to send a different type of wireless signal to the display unit or system 460B.

The power system 420B is configured to provide power to the detection system 410B. In the illustrated aspect, the power system 420B is operatively coupled to the detection system 410B and to the transmission system 430B. In some aspects, the power system 420B includes a battery, such as a zinc-air battery or other type of battery.

In the illustrated implementation, the housing 405B also houses a control system 440B. The control system 440B is operatively coupled to the detection system 410B, the power system 420B, and the transmission system 430B. The control system 440B is configured to control the device and the components of the device. For example, in some implementations, the control system 440B includes a memory and a processing system. In some implementations, an executable program is stored in the memory. In some implementations the control system 440B includes an analog front-end component.

The display unit 460B includes a receiving system 464B and a display 462B. The receiving system 464B is configured to receive signals from the transmission system 430B indicative of the blood pressure of the patient. For example, when the detection system 410B is operatively coupled to a vein of the patient, the receiving system 464B receives signals from the transmission system 430B indicative of the venous blood pressure of the patient. In some aspects, the receiving system 464B includes an antenna or other device that is configured to receive wireless signals. The receiving system 464B is operatively coupled to the display 462B. The display 462B is configured to visually display the measured blood pressure of the patient (such as the arterial blood pressure of the patient). For example, the display may be a light emitting diode display, a liquid crystal display, or another type of display. In some implementations the display unit 460B is configured to provide a visual indication as to what type of blood pressure is being displayed or which sensor the display unit 460B is receiving information from. For example, in some implementations the display 462B may provide such visual indication.

In some implementations, the sensor 401B includes an indicator 419B. The indicator 419B is configured to provide a visual indication as to which display system the sensor 401B is paired with. For example, the indicator 419B may include a light emitting diode or a series of light emitting diodes that provide a visual indication as to which display system the sensor 401B is paired with. In some examples, wireless communication and the indicator 419B allow a user or a physician to know what display the sensor 401B is communicating with without following a wire.

While the illustrated implementation includes two display units 460A and 460B, in some implementations, the system includes a single display unit. In such implementations, both sensors 401A and 401B are paired or otherwise in communication with the single display unit. The user or physician may select, for example, on the display unit which type of blood pressure is displayed.

In some implementations, the wireless communication of the device may be multiplexed to allow for the communication of multiple instruments to a single monitor. In some implementations, the sensors 401A and 401B are paired with a single display unit and operate within the same bandwidth without interference. In some implementations, the sensors 401A and 401B share the same bandwidth with other instruments such as an electrocardiogram (ECG) device, an SpO2 measurement device, a thermometer, or other type of medical device or medical instrument. In some implementations, signals of the sensors 401A and 401B and the other medical devices or medical instruments may be multiplexed and can communicate with a single monitor without interference.

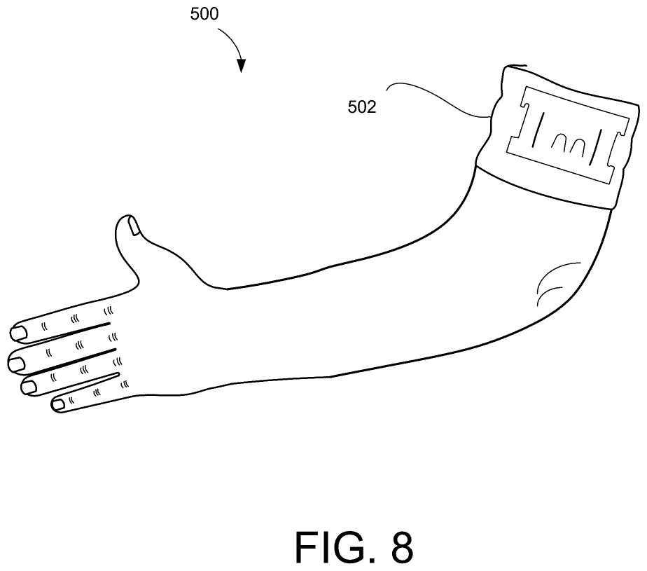

FIG. 8 illustrates a coupling member 500 that is operatively coupled to an arm of a patient. In the illustrated implementation, the coupling member 500 wraps around and is coupled to the upper arm of the patient. In other implementations, the coupling member 500 may be coupled to a different portion of the arm of the patient (such as the lower arm of the patient) or to a different portion of the body of the patient. The coupling member 500 includes an attachment plate or an attachment member 502. The attachment plate or the attachment member 502 is configured to be coupled to a sensor as described herein. In some examples, the attachment of the sensor to an upper arm of a patient helps ensure that the sensor is located at or near the level of the heart of the patient, which is desirable in some aspects. For example, in some cases the blood pressure measurements may be more accurate when the sensor is located at an elevation that is at or near the elevation of the heart of the patient.

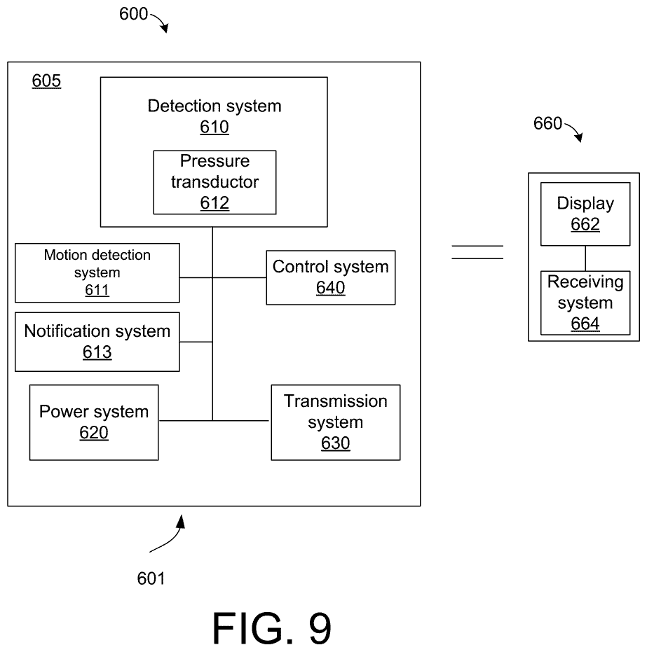

FIG. 9 schematically illustrates an apparatus 600 according to an implementation. The apparatus includes a sensor 601 and a display 660.

In some aspects, the apparatus 600 is configured to display or transmit for display, such as visually display, blood pressure measurements of a patient. Additionally, in some aspects the sensor 601 is configured to detect orientation or motion of the sensor 601 and to provide notification to the patient when there is an incorrect or non-ideal orientation of the sensor 601. For example, when the sensor 601 is coupled to an arm or other body portion of the patient, the patient may move the arm and cause the sensor 601 to be placed in a non-ideal orientation. For example, in some implementations, a non-ideal orientation may be when the elevation of the sensor is different than the elevation of the heart of the patient (for example, when the sensor is coupled to an arm of the patient and the patient raises or lowers the arm). In such cases, the sensor is configured to provide a notification to the patient, either a tactile notification, an audible notification, or another type of notification, that the sensor is in a non-ideal orientation.

The sensor 601 includes a detection system 610, the power system 620, the transmission system 630, and the control system 640 are disposed with the housing 605. For example, the housing 605 may define a cavity and the detection system 610, the power system 620, the transmission system 630, and the control system 640 are disposed within the cavity of the housing 605.

The detection system 610 is configured to detect or measure a blood pressure of a patient. In the illustrated aspect, the detection system 610 is an invasive blood pressure measuring system. Accordingly, the detection system 610 may be operatively coupled to an artery of a patient or to a vein of a patient. The detection system 610 includes a pressure transducer 612 that is configured to be operatively coupled to a patient, such as to an artery of the patient or to a vein of a patient, and to detect and measure the blood pressure of the patient.

The transmission system 630 is operatively coupled to the detection system 610. The transmission system 630 is configured to transmit a wireless signal indicative of the blood pressure of the patient as detected or measured by the detection system 610. For example, in some aspects, the transmission system 630 is configured to send a radiofrequency (RF) signal to a display unit or system 660. In other aspects, the transmission system 630 is configured to send a different type of wireless signal, such as a Bluetooth signal, to the display unit or system 660. In some aspects, the transmission system 630 includes a transmission antenna or other device that is configured to transmit wireless signals.

The power system 620 is configured to provide power to the sensor 601. In the illustrated aspect, the power system 620 is operatively coupled to the detection system 610 and to the transmission system 630. In some aspects, the power system 620 includes a battery, such as a zinc-air battery or other type of battery.

In the illustrated implementation, the housing 605 also houses a control system 640. The control system 640 is operatively coupled to the detection system 610, the power system 620 and the transmission system 630. The control system 640 is configured to control the device and the components of the device. For example, in some implementations, the control system 640 includes a memory and a processing system. In some implementations, an executable program is stored in the memory. In some implementations the control system 640 includes an analog front-end component.

The display unit 660 includes a receiving system 664 and a display 662. The receiving system 664 is configured to receive signals from the transmission system 630 indicative of the blood pressure of the patient. In some aspects, the receiving system 664 includes an antenna or other device that is configured to receive wireless signals. The receiving system 664 is operatively coupled to the display 662. The display 662 is configured to visually display the measured blood pressure of the patient. For example, the display may be a light emitting diode display, a liquid crystal display, or another type of display.

As illustrated in FIG. 9, the sensor 601 includes a motion detection system 611 and a notification system 613. The motion detection system 611 is configured to detect or determine the orientation and motion of the sensor 601. In some implementations, the motion detection system 611 includes an accelerometer that is configured to detect or determine the orientation or motion of the sensor 601.

The notification system 613 is configured to provide a notification to the patient when the sensor 601 is moved too much or is in a non-ideal orientation. For example, the notification system 613 may provide a tactile notification, an audible notification, or other type of notification to the patient.

Various implementations of the systems and devices disclosed herein, and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. Various implementations of the systems and techniques described here can be realized as and/or generally be referred to herein as a circuit, a module, a block, or a system that can combine software and hardware aspects. For example, a module or a system may include the functions/acts/computer program instructions executing on a processor (e.g., a processor formed on a silicon substrate, a GaAs substrate, and the like) or some other programmable data processing apparatus.

Specific structural and functional details disclosed herein are merely representative for the purposes of describing example aspects. Example aspects, however, be embodied in many alternate forms and should not be construed as limited to only the aspects set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example aspects. As used herein, the term and/or includes any and all combinations of one or more of the associated listed items.

The terminology used herein is for the purpose of describing particular aspects only and is not intended to be limiting of example aspects. As used herein, the singular forms “a”, “an”, and “the” are intended to include the plural forms as well, unless the context clearly indicates otherwise. It will be further understood that the terms comprises, comprising, includes and/or including, when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example aspects belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the above example aspects and corresponding detailed description are presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the above illustrative aspects, reference to acts and symbolic representations of operations that may be implemented as program modules or functional processes include routines, programs, objects, components, data structures, etc., that perform particular tasks or implement particular abstract data types and may be described and/or implemented using existing hardware at existing structural elements. Such existing hardware may include one or more Central Processing Units (CPUs), digital signal processors (DSPs), application-specific-integrated-circuits, field programmable gate arrays (FPGAs) computers or the like.

It should be borne in mind, however, that all of these and similar terms are to be associated with the appropriate physical quantities and are merely convenient labels applied to these quantities. Unless specifically stated otherwise, or as is apparent from the discussion, terms such as processing or computing or calculating or determining of displaying or the like, refer to the action and processes of a computer system, or similar electronic computing device, that manipulates and transforms data represented as physical, electronic quantities within the computer system's registers and memories into other data similarly represented as physical quantities within the computer system memories or registers or other such information storage, transmission or display devices.

Note also that the software implemented aspects of the example aspects are typically encoded on some form of non-transitory program storage medium or implemented over some type of transmission medium. The program storage medium may be magnetic (e.g., a floppy disk or a hard drive) or optical (e.g., a compact disk read only memory, or CD ROM), and may be read only or random access. Similarly, the transmission medium may be twisted wire pairs, coaxial cable, optical fiber, or some other suitable transmission medium known to the art. The example aspects not limited by these aspects of any given implementation.

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to employ the implementations in virtually any appropriately detailed structure. Further, the terms and phrases used herein are not intended to be limiting, but to provide an understandable description of the present disclosure.

It should also be noted that whilst the accompanying claims set out particular combinations of features described herein, the scope of the present disclosure is not limited to the particular combinations hereafter claimed, but instead extends to encompass any combination of features or aspects herein disclosed irrespective of whether or not that particular combination has been specifically enumerated in the accompanying claims at this time. Additionally, while certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the aspects.

What is claimed is:

1. An invasive blood pressure measuring apparatus, comprising:
a housing:
a detection system disposed within the housing, the detection system being configured to be operatively coupled to a patient and to measure a blood pressure of the patient; and
a transmission system disposed within the housing, the transmission system being operatively coupled to the detection system and configured to transmit a wireless signal indicative of a blood pressure of the patient.

2. The apparatus of claim 1, further comprising:
a power system disposed within the housing and being operatively coupled to the detection system and the transmission system.

3. The apparatus of claim 1, further comprising:
a power system disposed within the housing and being operatively coupled to the detection system and the transmission system, the power system including a battery.

4. The apparatus of claim 1, wherein the detection system includes a pressure transducer.

5. The apparatus of claim 1, wherein the pressure transducer is disposed adjacent a line that is configured to be fluidically coupled to an artery or a vein of the patient.

6. The apparatus of claim 1, wherein the transmission system includes a radiofrequency transmitter.

7. The apparatus of claim 1, wherein the housing includes a port, the port being configured to be coupled to a tubular member.

8. The apparatus of claim 1, wherein the housing includes a port, the port being configured to be operatively coupled to an artery or vein of the patient.

9. The apparatus of claim 1, wherein the housing includes a first port and a second port, the first port being configured to receive blood from an artery or a vein of the patient, the second port being configured to receive fluid from a fluid reservoir.

10. The apparatus of claim 1, further including:
an activation switch, the activation switch extending from the housing and being operatively coupled to the detection system and the transmission system.

11. The apparatus of claim 1, further including:
a power source disposed within the housing; and
an activation switch extending from the housing and being configured to activate the power source.

12. An invasive blood pressure measuring apparatus, comprising:
a housing defining a cavity:
a detection system disposed within the cavity of the housing, the detection system including a pressure transducer and being configured to measure a blood pressure of a patient;
a transmission system disposed within the cavity of the housing, the transmission system being operatively coupled to the detection system and configured to transmit a wireless signal indicative of a blood pressure of the patient;
a battery disposed within the cavity of the housing and operatively coupled to the detection system and to the transmission system;
an activation switch operatively coupled to the battery, at least a portion of the activation switch being disposed outside of the cavity of the housing; and
a coupling member configured to couple the housing to a portion of a body of the patient.

13. The apparatus of claim 12, wherein the pressure transducer is operatively coupled to an artery or vein of the patient.

14. The apparatus of claim 12, wherein the transmission system includes a radiofrequency transmitter.

15. The apparatus of claim 12, wherein the housing includes a port, the port being configured to be coupled to a tubular member.

16. The apparatus of claim 12, wherein the housing includes a port, the port being configured to be operatively coupled to an artery or vein of the patient.

17. The apparatus of claim 12, wherein the housing includes a first port and a second port, the first port being configured to be operatively coupled to an artery or vein of the patient, the second port being configured to be operatively coupled to a fluid reservoir.

18. A system, comprising:
an invasive blood pressure measuring device having,
a housing:
a detection system disposed within the housing, the detection system being configured to be operatively coupled to an artery or vein of a patient and to measure a blood pressure of the patient; and
a transmission system disposed within the housing, the transmission system being operatively coupled to the detection system and configured to transmit a wireless signal indicative of a blood pressure of the patient;
a coupling member configured couple the housing to a portion of a body of the patient; and
a display device having a transmission system and a display, the transmission system of the display device being configured to receive the wireless signal indicative of a blood pressure of the patient from the transmission system of the measuring device, the display being configured to display a blood pressure of the patient.

19. The system of claim 18, wherein the measuring device includes a power source disposed within the housing.

20. The system of claim 18, wherein the measuring device includes a power source and an activation switch, the power source being disposed within the housing, at least a portion of the activation switch being disposed outside of the housing.
